(19) United States
(12) Patent Application Publication
(10) Pub. No.: US 2012/0321732 A1
(43) Pub. Date: Dec. 20, 2012

(54) THERAPEUTIC USES OF TOMATO EXTRACTS

(75) Inventors: Niamh O'Kennedy, Windsor (GB); Hyun-Ju Song, Windsor (GB)

(73) Assignee: PROVEXIS NATURAL PRODUCTS LIMITED, Windsor (GB)

(21) Appl. No.: 13/456,025

(22) Filed: Apr. 25, 2012

Related U.S. Application Data
(63) Continuation of application No. 12/300,953, filed on Nov. 14, 2008, now abandoned.

(30) Foreign Application Priority Data
Jun. 2, 2006 (GB) 0610790.8

(51) Int. Cl. A61K 36/81; A61P 7/02

(52) U.S. Cl. 424/777

(57) ABSTRACT

The present invention relates to tomato extracts or an active fraction thereof for use in preventing or inhibiting the initiation of venous thrombosis and fibrin clot formation in a vein.

The present invention relates to compositions for use in the prophylaxis of venous thromboses.

### BACKGROUND TO THE INVENTION

Venous thromboses, the formation of blood clots in the veins, are responsible for large numbers of deaths each year, and represent a major worldwide health problem (Lopez, J. A., Kearon, C., Lee, A. Y. Y. (2004). Deep Venous Thrombosis. Hematology 2004: 439-456).

The main clinical conditions involving venous thromboses include deep vein thrombosis (DVT) and pulmonary embolism. DVT is a condition in which a blood clot develops in a deep vein, for example a deep vein in the leg or arm, or within the lower abdomen. It has been estimated that each year in the UK, about 1-3 people per 1000 in the UK develop DVT. In most cases of DVT, the clots are small and do not give rise to any symptoms. The body is able to break down the clots and there are no long term problems. In many cases, however, the clots are larger and consequently may partially or totally block blood flow in the vein leading to swelling of the muscle (e.g. calf muscle) surrounding the vein and pain in the muscle. In some cases, a piece of blood clot may break off and travel in the bloodstream to the lungs where it forms a pulmonary embolism blocking blood flow in the lungs, which can give rise to chest pain, shortness of breath or even, in severe cases, death.

There are well recognised risk factors that make DVT more likely to occur in an individual and these include advanced age, prolonged immobilisation, obesity, recent surgery, injuries such as fractures, the use of oral contraceptives, hormone replacement therapy, pregnancy, puerperium, cancer and treatments for cancer, antiphospholipid syndrome, various genetic risk factors, and plasmatic risk factors. Genetic risk factors, which are mainly related to the haemostatic system, include mutations in the genes that encode antithrombin, protein C and protein S, and the factor V Leiden and factor II G20210 A mutations. Plasmatic risk indicators include hyperhomocysteinemia and elevated concentrations of factors II, VIII, IX, XI and fibrinogen.

Thromboses occur because of a malfunction or inappropriate activation of components of the haemostatic system. The haemostatic system consists of two separate but linked systems; platelets and the coagulation proteins. Its primary function is to coordinate a network of molecular signals to ensure blood fluidity, while preventing blood loss. If vascular injury occurs, the integrity of the vascular system is maintained by the blood, which is converted into an insoluble gel at the site of injury in a process initiated by platelets and augmented by the coagulation proteins.

The mechanisms by which venous and arterial thromboses occur and the structures of the clots formed in the two types of thromboses differ significantly and, for this reason, venous and arterial thromboses are generally recognised as being distinct clinical entities.

In arterial thrombotic diseases such as atherosclerosis or events such as stroke or myocardial infarction, platelets are implicated in the initiation of thrombotic events. However, in conditions involving venous thrombosis, the onset of the condition is brought about by initiation of the coagulation cascade, and platelet aggregation plays a much less important role. Indeed, platelet aggregation inhibitors such as aspirin have been found to be of little use in preventing venous thrombosis.

Tissue factor (TF) is a transmembrane glycoprotein that is the major initiator of the coagulation cascade. During vascular injury, exposure of blood to subendothelial TF occurs. Exposed TF acts as a cofactor for the factor VIIa (FVIIa) catalysed activation of factor IX (FIX) and factor X (FX), critical components of the tenase and prothrombinase complexes, respectively. This leads to rapid formation of FXa and thrombin. Thrombin then cleaves fibrinogen to fibrin, which subsequently polymerises to form a fibrin clot.

The FVIIa/TF complex is involved in the pathogenic mechanism of a number of thrombotic diseases, and the circulating level of TF is a risk factor for thrombosis. Inappropriate exposure of blood to TF leads to chronic upregulation of circulating inflammatory cytokines, which in turn raise circulating levels of acute-phase inflammatory markers such as C-reactive protein. Disruption of the inflammatory system in this way can lead to TF expression on circulating monocytes, contributing to a sustained imbalance in the coagulation cascade, and spilling over into activation of the wider haemostatic system.

Although TF is released into the bloodstream by vascular injury, DVT can often arise in the absence of any damage to the walls of the veins. In recent years, evidence has accumulated indicating that TF circulates in normal plasma [see (1) Giesen et al. Blood-borne tissue factor: another view of thrombosis. Proc. Natl. Acad. Sci. USA. 1999; 96:2311-2315; (2) Koyama et al. Determination of plasma tissue factor antigen and its clinical significance. Br. J. Haematol. 1994; 87:343-347; and (3) Albrecht et al. Detection of circulating tissue factor and factor VII in a normal population. Thromb. Haemost. 1996; 75:772-777], both associated with cell-derived membrane micro-vesicles and as a soluble, alternatively spliced form. Endogenous TF-bearing micro-vesicles have been found to contribute to experimental thrombosis in vivo in the cremaster microcirculation (Falati et al. J Exp Med. 2003; 197:1585-1598), and have been shown to improve haemostasis in haemophilic mice (Hrachovinova et al. Nat. Med. 2003; 9:1020-1025). In the experimental systems described in the foregoing articles, TF-bearing microvesicles appeared to participate in thrombosis by binding to platelets or activated endothelial cells at sites of injury, a process dependent on the interaction between P-selectin glycoprotein ligand-1 (PSGL-1) on microvesicles and P-selectin on activated platelets.

International Patent application WO 99/55350 discloses the use of water-soluble extracts of tomato as inhibitors of platelet aggregation. The platelet aggregation inhibitory properties of the tomato extracts (known by the trade name Cardioflow® or Fruitflow™) have received considerable media publicity and it has been suggested in the popular press that the extracts, and tomato extracts from other sources, may reduce the risk of DVT—see for example (a) Vibrant Life, 1 Jan. 2006, ISSN: 0749-3509; Volume 22; Issue 1; (b) Main Report—Health and Wealth Letter, Drinking Tomato Juice Protects The Heart, 3 Oct. 2005; (c) Citywire, 18 Feb. 2005; (d) Verna Noel Jones, Chicago Tribune, RESOURCES. Q, 16 Jan. 2005; (e) Lindsay McIntosh, Aberdeen Press & Journal, 23 Sep. 2004; (f) The Express, 23 Sep. 2004, City And Business Ed. Stephen Kahn; (g) Citywire, 22 Sep. 2004; (h) The Sunday Mail, 5 Sep. 2004; (i) ANSA—English Media Service, HEALTH: TOMATOES CAN PREVENT HEART DISEASES, 28 Aug. 2004; and similar articles.

In each of the foregoing articles where the underlying basis for the properties of the extracts is discussed, speculation regarding the potential use of the extracts in reducing the risk of DVT is invariably based on the known platelet aggregation inhibitory activities of the extracts. However, as discussed above, platelet aggregation is not responsible for initiating venous thrombosis, and platelet aggregation inhibitors such as aspirin have been found to be of little use in preventing DVT. The reports in the popular media that tomato extracts can prevent DVT by virtue of their platelet aggregation inhibitory properties are put in perspective by a contemporaneous article in the medical journal GP (Haymarket Publications, London, UK), 5 Apr. 2004, entitled “GP Clinical—Behind The Headlines—Can tomato drink halt blood clots?”. In the article, the authors concluded that General Practitioners should tell their patients that—“The drink may not help prevent DVT as anti-platelet agents do not have much impact in the venous system”.

Therefore, as far as the applicants are aware, there is no evidence in the literature to date to suggest that tomato extracts have any benefit in treating venous thrombosis. Moreover, there has been no suggestion in the literature that tomato extracts have any effect on the coagulation cascade that initiates the formation of venous blood clots.

### SUMMARY OF THE INVENTION

It has now been found that events mediated by Tissue Factor (TF) are affected by water-soluble extracts of tomato and it has also been found that the extracts can reduce in vitro clotting times in blood, plasma (from which blood cells including blood platelets have been removed). The results obtained so far indicate that the tomato extracts should be useful in preventing the initiation of venous thrombosis.

Accordingly, in a first aspect, the invention provides a tomato extract or an active fraction thereof for use in preventing or inhibiting the initiation of venous thrombosis.

In another aspect, the invention provides a tomato extract or an active fraction thereof for use in preventing or inhibiting the initiation of fibrin clot formation in a vein.

The term “active fraction” as used herein refers to a fraction isolated from a tomato extract, which fraction has the ability to prevent the initiation of fibrin clot formation in a vein or to prevent the initiation of venous thrombosis.

The invention also provides;
The use of a tomato extract or an active fraction thereof for the manufacture of a medicament for preventing or inhibiting the initiation of venous thrombosis.
The use of a tomato extract or an active fraction thereof for the manufacture of a medicament for preventing or inhibiting the initiation of fibrin clot formation in a vein.
A composition comprising a tomato extract or an active fraction thereof for use in preventing or inhibiting the initiation of venous thrombosis.
A composition comprising a tomato extract or an active fraction thereof for use in preventing or inhibiting the initiation of fibrin clot formation in a vein.
A method of preventing or inhibiting the initiation of venous thrombosis in a mammal such as human, which method comprises administering to the mammal an effective amount of a tomato extract or active fraction thereof.
A method of preventing or inhibiting the initiation of fibrin clot formation in a vein, which method comprises administering to the patient an effective amount of a tomato extract or active fraction thereof.

The tomato extracts of the invention may be directed to preventing or inhibiting the initiation of venous thrombosis (or inhibiting or preventing the initiation of a fibrin clot in a vein) in a patient who is at greater than normal risk of suffering an occurrence of venous thrombosis by virtue of belonging to any one or more (in any combination) of the following at-risk sub-populations:
patients of an age greater than 50, for example greater than 60, or greater than 70, or greater than 80;
patients who are subjected to prolonged immobilisation, for example for a period of more than 3, 4, 5, 6, 7, 8, 9, 10, 11 or 12 hours, or more than 1, or 2, or 3, or 4, or 5 days;
patients who are clinically obese;
patients who have recently undergone surgery (for example in the past month, or the past 21 days, or 14 days, or 7 days);
patients suffering from injuries such as fractures;
patients taking oral contraceptives;
patients being treated with hormone replacement therapy;
patients who are pregnant;
mothers who have recently given birth (puerperium);
patients suffering from cancer and patients receiving treatments for cancer;
patients suffering from antiphospholipid syndrome;
patients possessing a genetic risk factor; and
patients possessing a plasmatic risk factor.

For patients possessing a genetic risk factor, the risk factor can be any one or more (in any combination) of the following:
a mutation in the gene that encodes antithrombin;
a mutation in the gene that encodes protein C;
a mutation in the gene that encodes protein S;
a factor V Leiden mutation; and
a factor II G20210 A mutation.

For patients possessing a plasmatic risk factor, the risk factor can be any one or more (in any combination) of the following:
hyperhomocysteinemia;
an elevated concentration of factor II;
an elevated concentration of factor VIII;
an elevated concentration of factor IX;
an elevated concentration of factor XI; and
an elevated level of fibrinogen.

Accordingly, in another aspect, the invention provides a tomato extract or active fraction thereof for use in the prophylaxis of venous thrombosis in a patient in any one or more (in any combination) of the at-risk sub-populations as defined herein.

The invention also provides the use of a tomato extract or active fraction thereof for the manufacture of a composition (e.g. medicament) for the prophylaxis of venous thrombosis in a patient in any one or more (in any combination) of the at-risk sub-populations as defined herein.

The invention further provides a method for the prophylaxis of venous thrombosis in a patient (e.g. a mammalian patient such as a human patient) in any one or more (in any combination) of the at-risk sub-populations as defined herein, which method comprises administering to the patient an effective amount of a tomato extract or active fraction thereof.

In a further aspect, the invention provides a use, extract for use, method or composition for use, wherein the patient is a member of a sub-population of people that suffer from recurring venous thrombosis, such as recurring deep vein thrombosis.

The term “effective amount” as used herein refers to an amount that confers a therapeutic effect on a patient. The therapeutic effect may be objective (i.e. measurable by some test or marker) or subjective (i.e., patient gives an indication of or feels an effect).

Further aspects and embodiments of the invention are as set out below and in the claims appended hereto.

The tomato extracts of the invention have been found to act in several ways to prevent or inhibit the activity of Tissue Factor (TF).

Firstly, tomato extracts have been found to influence plasma clotting times (PT, TCT, aPTT) thereby implying potential mediation of clotting factors.

Secondly, the tomato extracts have been found to reduce the expression of p-selectin on activated platelets. As discussed above, the binding of tissue-factor bearing microvesicles to p-selectin on the surfaces of platelets and endothelial cells is believed to play a part in the initiation of the coagulation cascade.

Thirdly, the extracts have been found to block the interaction of TF with the PAR2 receptor on the surface of human umbilical vein endothelial cells (HUVEC cells). The PAR2 receptor is a substrate for the TF/FVIIa complex and FXa.

On the basis of the three findings set out above, it is considered that the tomato extracts and active fractions of the invention will be useful in inhibiting the initiation of venous thrombosis and the formation of fibrin clots.

### Preparation and Characterisation of the Extracts

Whereas whole tomatoes or tomatoes that have been chopped or otherwise comminuted but not fractionated may be used for the purposes of the invention, it is preferred to use aqueous extracts of the tomato.

Such extracts may be prepared by homogenising the flesh of a tomato, with or without its skin, and then filtering the homogenate to remove solids. Preferably, substantially all water-insoluble solids are removed, for example by centrifugation and/or filtration.

Alternatively, commercially available tomato pastes may be used as the starting material for the preparation of the extracts. The tomato pastes are typically diluted with water, and then water-insoluble solids are removed, e.g. by centrifugation and/or filtration to give a substantially clear solution.

In each case, removal of the solids has the effect of removing fragments of skin containing lycopene. Thus, the preferred tomato extracts of the invention are water soluble extracts that are substantially free of lycopene.

The aqueous filtrate may be subjected to further fractionation to provide an active fraction containing a compound or compounds responsible for the biological or therapeutic effects described herein. Alternatively, the filtrate may be evaporated to give a dry water soluble extract.

Filtration of the tomato homogenate may be accomplished in a single stage, or in a series of filtration steps, starting with a relatively coarse filtration or centrifugation step to remove larger particles of tomato skin and/or other water-insoluble fragments of tomato flesh. Further filtration steps may then be effected to give a substantially clear solution, e.g. a solution that will pass through a 0.2μ filter without loss of solids.

Thus, in one preferred embodiment of the invention, the tomato extract is a water soluble extract substantially free of lycopene and capable of passing through a 0.2μ filter without loss of solids.

In one embodiment, native sugars are removed from the tomato extracts. An advantage of removing the sugars is that the activity of the extracts is concentrated and the extracts tend to be less sticky and easier to process in the solid form.

Where the starting material for the preparation of the extracts is a tomato paste, it is preferably one that has been produced by means of a “cold-break” process rather than a “hot-break” process. The terms “cold-break” and “hot-break” are well known in the field of tomato processing and commercially available tomato pastes are typically sold as either hot-break or cold-break pastes. Cold-break pastes can be prepared by a process involving homogenisation of the tomato followed by a thermal processing step in which the tomatoes are heated to temperature of no more than about 60° C., in contrast to hot-break pastes where the homogenised tomatoes are subjected to thermal processing at temperatures of about 95° C., see for example, Anthon et al., J. Agric. Food Chem. 2002, 50, 6153-6159.

In an alternative method, an aqueous extract of tomatoes or tomato paste can be produced by enzyme digestion of pectins and starch in homogenised fruit or paste, followed by removal of suspended solids from the homogenate, and micro- or ultrafiltration to remove large molecular weight proteins and remaining polysaccharides. The extract can be refined by removal of simple sugars, for example glucose, fructose and sucrose, leaving a concentrated water-soluble extract which contains a wide variety of low molecular weight (<1000 Da) non-sugar tomato components. Removal of simple sugars can be carried out by crystallisation, for example using low-temperature ultrasound-assisted crystallisation, or ethanol precipitation of crystalline glucose and fructose. Alternatively, simple sugars can be separated from other extract components by a chromatographic procedure, for example selective adsorption of bioactive extract components from the aqueous solution onto a polystyrene-based resin material, allowing selective removal of glucose, fructose and sucrose in the waste stream. The adsorbed non-sugar compounds are then recovered from the adsorbent resin material by elution with ethanol, followed by removal of the ethanol by evaporation. The non-sugar components can be dried into a water-soluble powder by spray drying or drum drying, or alternatively resuspended in water to give an aqueous syrup. Aqueous extracts prepared in this way represent a further aspect of the invention.

Sugar-free tomato extracts, for example those discussed above, typically contain a variety of compounds of molecular weight<1000 Da and represent preferred extracts for use according to the invention.

The inventor has also found that the tomato extracts are efficacious if they have no or low nucleoside content. Accordingly preferred extracts have no or less that 10 nM nucleosides contained therein.

The tomato extracts of the invention comprise a number of bioactive components. Preferred extracts contain bioactive components selected from: phenolic compounds, amino acids and amino acid conjugates, and tomato flavour compounds.

The extract preferably comprises phenolic compounds selected from flavonoids and flavonoid derivatives, for example derivatives of quercetin, kaempferol and naringenin; hydroxycinnamic acids and derivatives, for example ferulic acid, coumaric acid and their conjugates; benzoic acids and derivatives such as benzoic acid, hydroxybenzoic acid, gallic acid, salicylic acid and conjugates.

The extract preferably comprises amino acids selected from tyrosine and hydroxytyrosine, phenylalanine, glutamine, and their conjugates.

The flavour compounds may be selected from hexanal derivatives, dimethylsulfide, b-damascenone, 3-methylbutyric acid, eugenol and methional.

The tomato extracts may be sub-fractionated by HPLC to give three subfractions, AF1, AF2 and AF3, on the basis of polarity. In the experimental section below, the unfractionated extract (tAF) and the fractions AF1-3 have then been used to carry out in vitro experiments.

### Pharmaceutical and Nutraceutical Formulations

The extracts or active fractions thereof may be formulated for oral administration. As such, they can be formulated as solutions, suspensions, syrups, tablets, capsules, lozenges and snack bars, inserts and patches by way of example. Such formulations can be prepared in accordance with methods well known per se.

For example, the extracts or active fractions can be formed into syrups or other solutions for administration orally, for example health drinks, in the presence of one or more excipients selected from sugars, vitamins, flavouring agents, colouring agents, preservatives and thickeners.

Tonicity adjusting agents such as sodium chloride, or sugars, can be added to provide a solution of a particular osmotic strength, for example an isotonic solution. One or more pH-adjusting agents, such as buffering agents can also be used to adjust the pH to a particular value, and preferably maintain it at that value. Examples of buffering agents include sodium citrate/citric acid buffers and phosphate buffers.

Alternatively, the extracts or active fractions thereof can be dried, e.g. by spray drying or freeze drying, and the dried product formulated in a solid or semi solid dosage form, for example as a tablet, lozenge, capsule, powder, granulate or gel.

Simple dried extracts can be prepared without any additional components. Alternatively, dried extracts can be prepared by adsorbing on to a solid support; for example a sugar such as sucrose, lactose, glucose, fructose, mannose or a sugar alcohol such as xylitol, sorbitol or mannitol; or a cellulose derivative. Other particularly useful adsorbents include starch-based adsorbents such as cereal flours for example wheat flour and corn flour. For tablet formation, the dried extract is typically mixed with a diluent such as a sugar, e.g. sucrose and lactose, and sugar alcohols such as xylitol, sorbitol and mannitol; or modified cellulose or cellulose derivative such as powdered cellulose or microcrystalline cellulose or carboxymethyl cellulose. The tablets will also typically contain one or more excipients selected from granulating agents, binders, lubricants and disintegrating agents. Examples of disintegrants include starch and starch derivatives, and other swellable polymers, for example crosslinked polymeric disintegrants such as cross-linked carboxymethylcellulose, crosslinked polyvinylpyrrolidone and starch glycolates. Examples of lubricants include stearates such as magnesium stearate and stearic acid. Examples of binders and granulating agents include polyvinylpyrrolidone. Where the diluent is not naturally very sweet, a sweetener can be added, for example ammonium glycyrrhizinate or an artificial sweetener such as aspartame, or sodium saccharinate.

Dried extracts can also be formulated as powders, granules or semisolids for incorporation into capsules. When used in the form of powders, the extracts can be formulated together with any one or more of the excipients defined above in relation to tablets, or can be presented in an undiluted form. For presentation in the form of a semisolid, the dried extracts can be dissolved or suspended in a viscous liquid or semisolid vehicle such as a polyethylene glycol, or a liquid carrier such as a glycol, e.g. propylene glycol, or glycerol or a vegetable or fish oil, for example an oil selected from olive oil, sunflower oil, safflower oil, evening primrose oil, soya oil, cod liver oil, herring oil, etc. Such extracts can be filled into capsules of either the hard gelatine or soft gelatine type or made from hard or soft gelatine equivalents, soft gelatine or gelatine-equivalent capsules being preferred for viscous liquid or semisolid fillings.

Dried extracts can also be provided in a powder form for incorporation in to snack food bars for example fruit bars, nut bars, and cereal bars. For presentation in the form of snack food bars, the dried extracts can be admixed with any one or more ingredients selected from dried fruits such as sun-dried tomatoes, raisins and sultanas, groundnuts or cereals such as oats and wheat.

Dried extracts can be provided in a powder form for reconstitution as a solution. As such they can also contain soluble excipients such as sugars, buffering agents such as citrate and phosphate buffers, and effervescent agents formed from carbonates, e.g. bicarbonates such as sodium or ammonium bicarbonate, and a solid acid, for example citric acid or an acid citrate salt.

In one preferred embodiment, dried extract is provided in powder form optionally together with a preferred solid (e.g. powdered) excipient for incorporation into capsules, for example a hard gelatine capsule.

In another embodiment, the dried extract is one from which substantially all native sugars have been removed.

A solid or semisolid dosage form of the present invention can contain up to about 1000 mg of the dried extract, for example up to about 800 mg.

The extracts can be presented as food supplements or food additives, or can be incorporated into foods, for example functional foods or nutraceuticals.

The compositions of the invention can be presented in the form of unit dosage forms containing a defined concentration of extract or active fraction thereof. Such unit dosage forms can be selected so as to achieve a desired level of biological activity. For example, a unit dosage form can contain an amount of up to 1000 mg (dry weight) of an extract or active fraction, more typically up to 800 mg, for example 50 mg to 800 mg, e.g. 100 mg to 500 mg. Particular amounts of extract or active fraction that may be included in a unit dosage form may be selected from 50 mg, 100 mg, 150 mg, 200 mg, 250 mg, 300 mg, 350 mg, 400 mg, 450 mg, 550 mg, 600 mg, 650 mg, 700 mg, 750 mg and 800 mg.

The compositions of the invention can be included in a container, pack or dispenser together with instructions for administration.

### Pharmaceutical Uses

For use in preventing or inhibiting the initiation of venous thrombosis, the quantity of extract or active fraction administered to a patient per day will depend upon the strength of the extract and the particular condition or disease under treatment and its severity, and ultimately it will be at the discretion of the physician. The amount administered however will typically be a non-toxic amount effective to bring about the desired result.

For example, a typical daily dosage regime for a human patient potentially at risk of suffering from venous thrombosis may be from 0.0001 to 0.1, preferably 0.001 to 0.05 gram per kilogram body weight. When an active fraction is isolated and administered, the amount of solid material administered can be reduced by an amount consistent with the increased purity of the fraction. Typically, at least 100 mg (dry weight or dry weight equivalent) and preferably at least 200 mg, and more usually at least 500 mg of the extract will be administered per day to a human patient.

The compositions can be administered in single or multiple dosage units per day, for example from one to four times daily, preferably one or two times daily.

The extracts of the invention can be administered in solid, liquid or semi-solid form. For example, the extracts can be administered in the form of tomato juice or concentrates thereof alone or in admixture with other fruit juices such as orange juice.

### BRIEF DESCRIPTION OF THE DRAWINGS

The invention will now be illustrated, but not limited, by the following example, and with reference to the accompanying drawings, in which:—

FIG. 1 is a HPLC chromatogram showing the subfractionation of a tomato extract of the invention (tAF) into three subfractions AF1-AF3 as discussed in Example 2.

FIG. 2 shows, as discussed in Example 3, the effects of AF1-AF3 on clotting time parameters in vitro. Subfractions of tAF (AF1-AF3) were preincubated with plasma prior to initiation of clotting with PT, TCT or aPTT reagents. All inhibitor solutions were used at a final concentration of 0.08 g/L; for all measurements, n=3.

FIG. 3 shows, as discussed in Example 4, the inhibition of ADP-induced expression of the platelet activation marker p-selectin, after pre-incubation of whole blood with the tomato extract active fraction, tAF, and its subfractions AF1-AF3. All inhibitors were used at a final concentration of 0.05 g/L; for each measurement, n=5. All inhibitors showed significant differences from control (P<0.001).

FIG. 4 shows, as discussed in Example 4, the dose response relationship observed for the inhibition of p-selectin expression in activated platelets by tAF (shown in FIG. 3).

FIG. 5 demonstrates that pre-incubation with the tomato extracts of the invention reduces the levels of interleukin-6 and interlueukin-8 generated by human umbilical vein endothelial cells (HUVEC cells). Significant differences from control (con −) are shown on the graphs and are discussed in Example 5.

### EXAMPLE 1

### Preparation of a Tomato Extract

A tomato extract for use in the therapeutic method of the invention was prepared using commercially available cold-break tomato paste of 28-30° Brix (i.e. 28-30% solids, w/w) having a browning index<0.350 AU (where browning index is defined as absorbance of a solution of concentration 12.5 g soluble solids/L at 420 nm) as the starting material. The paste was diluted (˜1:5) with ultrapure water and large particulate matter was removed by centrifugal filtration followed by clarification using a Westfalia MSB-14 Separator (a centrifugal disc clarifier) at room temperature. Smaller particulate matter was then removed by microfiltration at a temperature not exceeding 45° C., to give a clear straw-coloured solution containing no insoluble (spin-down) solids and capable of passing through a 0.2μ filter without loss of soluble solids. This solution was concentrated by evaporation to a syrup of 62-65° Brix, using carefully controlled conditions and a temperature not exceeding 80° C. to limit the progress of non-enzymic browning reactions. A flash pasteurisation step (T=105° C. for 3 seconds) was incorporated at the outset of the evaporation procedure. The final product was characterised by a browning index<0.600 AU, and a microbial total plate count of <1000.

The concentrated extract may be added to an orange juice matrix for administration to patients.

### EXAMPLE 2

### Alternative Preparation of Tomato Extract and Sub-Fractions Thereof

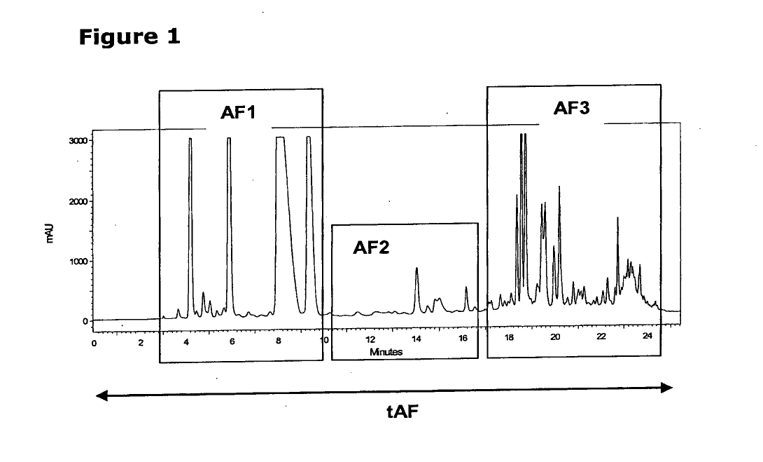

An aqueous extract from ripe tomato fruit was prepared by homogenization of fresh tomatoes (Lycopersicon esculentum, sourced locally), centrifugation, and clarification of the resulting straw-coloured liquid by ultrafiltration (ultrafiltration membrane, MW cut-off 1000 Da, Millipore (UK) Ltd., Watford, UK). Analysis showed that the tomato aqueous extract consisted largely of soluble sugars (85-90% of dry matter). These constituents were removed using solid phase extraction with styrene divinylbenzene (SDVB) cartridges (J T Baker, Mallinckrodt Baker B V, Deventer, Holland) at pH 2.5. Non-sugar components were retained on the cartridges and eluted in methanol. Non-sugar material isolated (the total active fraction, tAF) accounted for approximately 4% of the aqueous extract dry matter. Semi-preparative HPLC was used to subfractionate tAF components into three broad groups (Synergy Polar-RP, 4μ, 250×10 mm and Luna C18(2), 3μ, 250×10 mm columns, Phenomenex, Macclesfield, UK: Acetonitrile/0.05% TFA gradients). These three groups, labelled ‘AF1’, ‘AF2’ and ‘AF3’ in order of decreasing polarity, are shown on the HPLC chromatogram in FIG. 1. The isolated tAF and sub-fractions AF1-AF3 were reconstituted to known concentrations in phosphate-buffered saline (PBS, Sigma-Aldrich, Poole, UK), and solution pH was adjusted to 7.4 before use in in vitro experiments.

### EXAMPLE 3

### Inhibition of Clotting Times by Components in Tomato Extracts

it has previously been shown that tomato extract components inhibit platelet aggregation in vitro and ex vivo (see our earlier patent application WO 99/55350). Platelets represent one part of the haemostatic system, working in tandem with the coagulation cascade to balance blood fluidity and blood clotting. This experiment was designed to examine whether tomato extract could also affect the coagulation cascade, independently from its known effects on blood platelets. Plasma clotting times (measured in plasma from which platelets have been removed) are used to provide measurements reflecting the status of the coagulation cascade, independent of platelet function. Prothrombin and thrombin clotting times (PT and TCT respectively) are used to provide specific measures of the adequacy of the extrinsic system, incorporating the clotting abilities of factors I (fibrinogen), II (prothrombin), V, VII and X. Tissue factor must be added from an external source to allow the extrinsic system to function. Activated partial thromboplastin time (aPTT) is used to define the intrinsic system, and examines the clotting abilities of factors XII, XI, IX, V and VIII, all of which are normally present in plasma.

Experimental Details:

Clotting time measurements were performed on a CoaData 4001 coagulometer (Helena Biosciences, UK), following procedures as specified by the manufacturer. Briefly, PT, TCT and aPTT reagents, together with Norm-Trol quality control plasma, were obtained from Helena Biosciences, and the coagulometer calibrated to % PT/TCT/aPTT using a calibrant kit from the same supplier. Citrated plasma warmed to 37° C. was incubated with either control (saline) or treatment (see below) solutions for 15 minutes. The treated plasma was then incubated with PT/TCT/aPTT reagent, with stirring, and the time taken before clot formation was recorded in duplicate by the coagulometer. Duplicate measurements with a coefficient of variance of less than 5% were accepted. Norm-Trol QC plasma was used as a control.

Treatments:

Tomato extracts AF1, AF2, AF3 as described in Example 2 were used as treatments. All treatment solutions were made up to give a final concentration of 80 μg/mL plasma. Physiological saline (0.9% NaCl) was used as a control. All control and treatment solutions were adjusted to pH 7.4 and warmed to 37° C. before use.

Results:

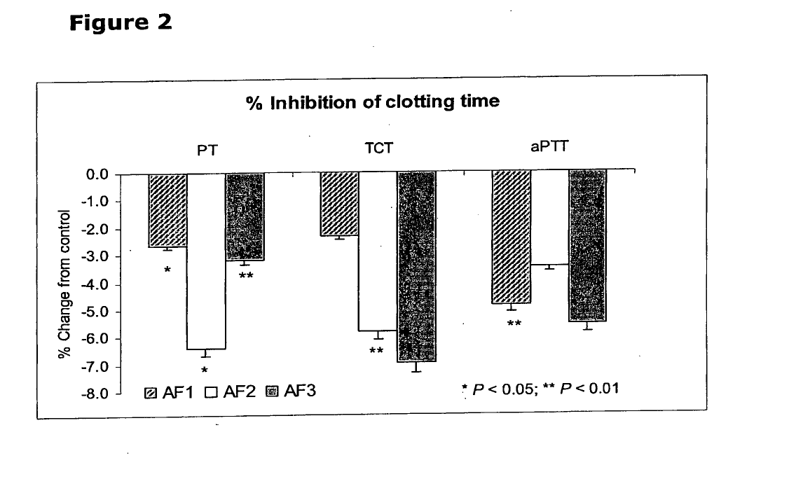

Data obtained showed an increase in the amount of time over which clot formation occurred in the treated plasma, compared to control plasma. FIG. 2 shows the results obtained, expressed as % inhibition of clotting time (s), compared to control values. Tomato extract fractions AF1 and AF2 showed the most significant effects on clotting time parameters. The extrinsic pathway (measured by PT and TCT) was more strongly affected than the intrinsic pathway.

Conclusions:

The results obtained show that in vitro, some components of the tomato extract are capable of interaction with the blood factors which together make up the coagulation cascade, and this effect is of interest when considering the overall function of the haemostatic system. The increased effects on the extrinsic system suggest that tissue factor-mediated pathways of coagulation, known to be of particular importance in venous thrombosis, could be beneficially suppressed by the use of the tomato extracts of the invention.

### EXAMPLE 4

### Investigation into the Effect of Tomato Extract on ADP-Induced Platelet P-Selectin Expression

The following experiment was designed to further explore mechanisms by which blood coagulation can be altered by tomato extract components. Platelet activation results in release of pro-coagulant signalling molecules from the surface of the activated platelet. Subsequent interaction with the blood vessel wall results in activation of the coagulation cascade. One of the most prominent of these pro-coagulant platelet-derived signalling molecules is p-selectin. In this experiment, we measured the expression of p-selectin on the surface of activated platelets, using a fluorescence-tagged antibody to p-selectin. The level of p-selectin derived fluorescence was quantified by flow cytometry. Effects of tomato extract components on p-selectin expression were then quantified.

Experimental Details:

Freshly drawn whole blood diluted 1:10 with HEPES-Mg buffer (pH 7.4) was preincubated with treatment solutions (see below) or with control solution (HEPES-Mg buffer) for 10 minutes. To induce p-selectin expression on platelets, aliquots (40 μL) of these mixtures were then incubated with or without ADP (final concentration 3 μmol/L) in Falcon polystyrene tubes (BD Biosciences, Cowley, UK) for 5 minutes at room temperature. 10 μL of a saturating concentration of two fluorescence-tagged monoclonal antibodies was then added to the incubation tubes. Fluoroscein isothiocyanate-labelled anti-CD61 (anti-CD61-FITC) was added to positively identify all platelets in the test samples (CD61 is a platelet-specific protein, not expressed by other blood cells). Phycoerythrin-labelled anti-p-selectin (anti-CD62P-PE) was added to bind to expressed p-selectin on the platelet surface. FITC & PE labeled mouse IgG antibodies were used as isotype controls. Incubation proceeded for 20 minutes in the dark at room temperature. Ice-cold phosphate-buffered saline (2 mL) was then added, and the samples were analyzed on a FACSCalibur flow cytometer with CellQuest software (BD Biosciences, Cowley, UK). Activated platelets were defined as the percentage of CD61-positive events co-expressing the CD62P receptor.

Treatments:

Tomato extracts tAF and AF1-AF3 as described in Example 2 were used as treatments. All treatment solutions were made up to give a final concentration of 50 μg/mL. HEPES-Mg buffer was used as a control. All control and treatment solutions were adjusted to pH 7.4 and warmed to 37° C. before use. For each measurement, n=5.

Results:

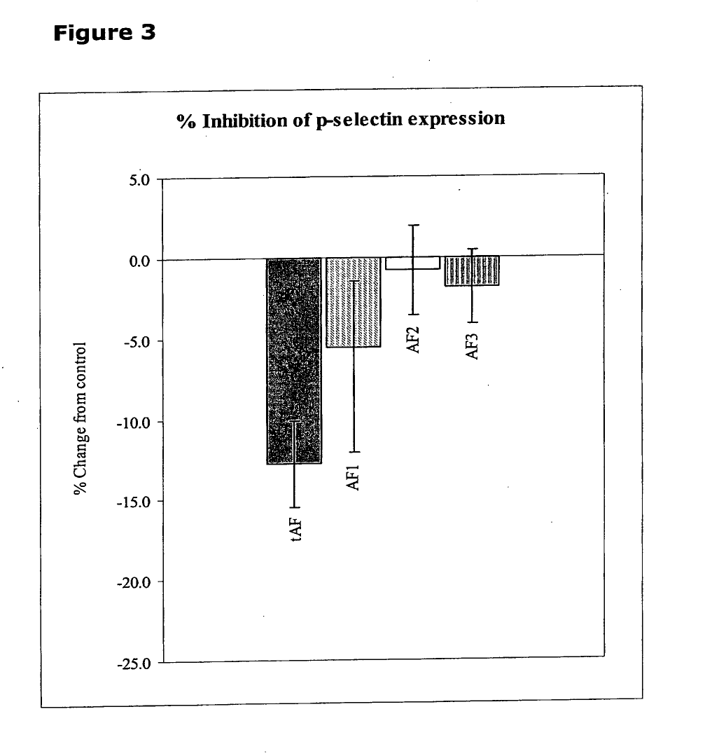

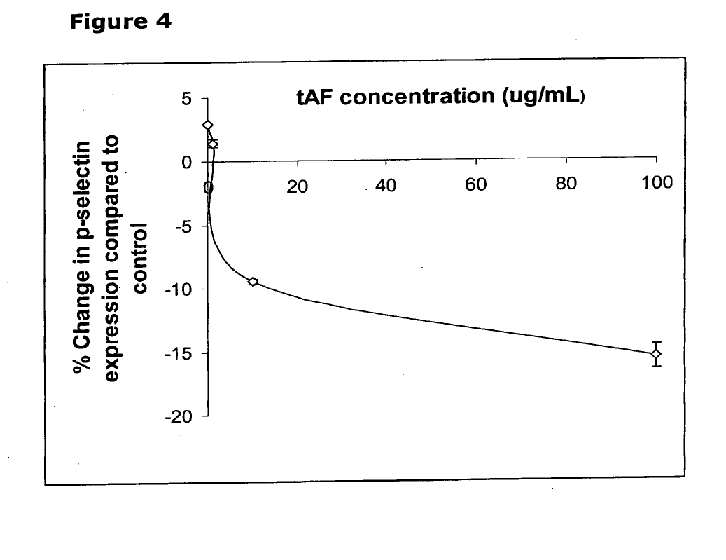

On stimulation with 3 μmol/L ADP, 41.2-68.2% p-selectin positive platelets were recorded in control samples, with a median value of 51.1%. The experimental variance was below 5%. Pre-incubation of diluted whole blood with tAF and AF1-AF3 resulted in a significant inhibition of activation-induced p-selectin expression, compared to control values (P<0.001, FIG. 3). The effects of tAF on p-selectin expression was significantly different from the effects of each of the subfractions AF1-AF3, although no differences were detected between the individual subfractions. A dose response was observed in the inhibition of p-selectin by tAF (range 0-100 μg/mL final concentration, FIG. 4).

Conclusions:

The results obtained show that in vitro, tomato extracts prevent expression of p-selectin on the platelet surface, reducing platelet activation in response to ADP agonist. This effect is of considerable significance to the coagulation cascade. Expression of p-selectin on activated platelets leads to release of p-selectin into the bloodstream (soluble p-selectin, or sP-selectin), leading to a source of the protein which can persist after platelet activation has been suppressed. Higher levels of soluble p-selectin are linked to venous thrombotic diseases, due to its function as a mediator of binding between platelets and monocytes, and its interaction with tissue factor to provide a link between platelet activation and the coagulation cascade. Our results suggest that this link to the coagulation cascade can be broken due to the positive effects of tomato extracts on p-selectin expression; thus tomato extract, by reducing both platelet-bound and circulating p-selectin, can potentially reduce the risk of venous thrombosis.

### EXAMPLE 5

### Investigation into the Effect of Refined Tomato Extract and its Sub-Fractions on TF-Induced Cytokine Release in Cultured Human Umbilical Vein Endothelial Cells (HUVEC Cells)

Example 4 demonstrates that tomato extract could potentially be beneficial in preventing the initiation of venous thrombosis due to its suppression of p-selectin expression on platelets. P-selectin is an integral part of tissue factor mediated initiation of the coagulation cascade, a pathway thought to be responsible for the initiation phase of venous thrombosis. We hypothesised that by reducing p-selectin expression on the surface of activated platelets or endothelial cells, tomato extract can prevent tissue factor-bearing microvesicles from adhering to endothelial cells, and thus prevent the close contact which is necessary for thrombin generation and clot formation.

To demonstrate that the effects of tissue factor on endothelial cells can be inhibited in the presence of tomato extract, we designed an experiment involving human umbilical vein endothelial cells (HUVEC cells). HUVEC cells express the protease activated receptors PAR1 and PAR2. PAR2 is a substrate for TF/FVIIa and FXa, and when activated stimulates release of pro-inflammatory cytokines IL-6 and IL-8. Thus by measuring TF-mediated generation of IL-6 and IL-8, the effects of pre-incubation with tomato extract components can be assessed.

Experimental Details:

HUVEC cells were grown (up to passage 5) in BGM-2 medium. Cells were serum-starved for 5 hours, then treated with 25 nM TF/10 nM FVIIa and 100 nM FX, in the presence or absence of tomato extract components (treatments explained fully below). Treated cells were incubated for 20 hours, after which time supernatants were collected and frozen at −80° C. until analysis for IL-6 and IL-8 by ELISA.

Treatments:

Concentration of tomato extract active components (tAF as described in Example 2) used as the ‘base’ treatment was calculated as the maximum concentration achievable in the circulation of a normal individual (blood volume 5.5 L) consuming 2.5 fresh tomatoes, assuming full absorption of all components. For tAF, this quantity was 43 mg/L. Three sub-fractions of tAF, AF1-AF3 as described in Example 2, were also tested, at concentrations which reflected their contribution to tAF on a dry-weight basis, i.e. 13.6 mg/L, 5.5 mg/L and 23.4 mg/L for AF1, AF2 and AF3 respectively. All four treatments were tested at these base concentrations, and also at twice and tenfold the base concentration. Saline was used as a control treatment.

Results:

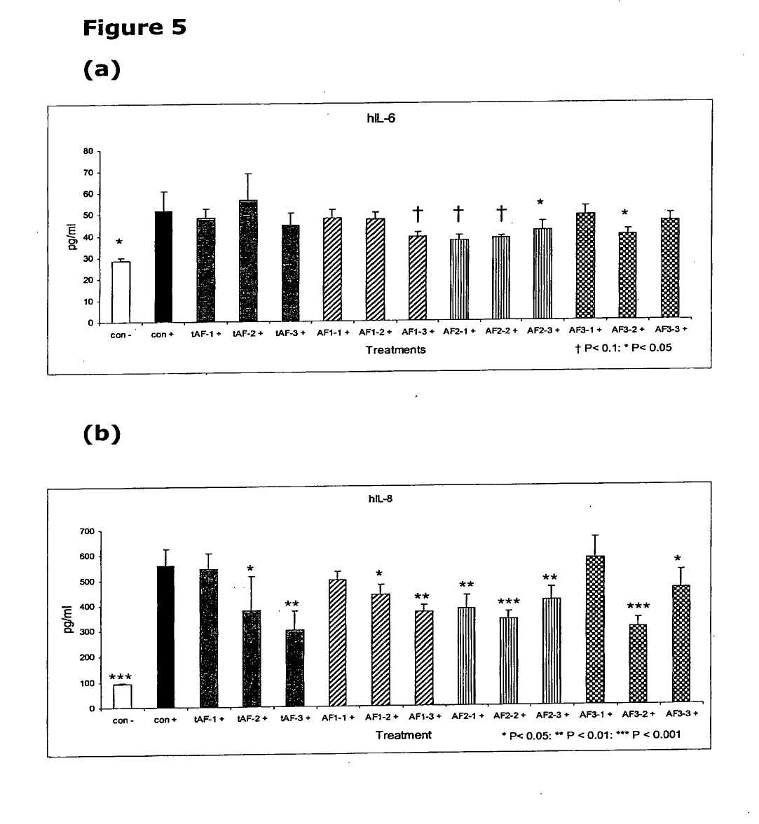

Pre-incubation with tomato extract components reduced levels of IL-6 generated by HUVECs by up to 12%, and IL8 by 10-50% (see FIG. 5). This implies that the components of tomato extract known to inhibit p-selectin expression also reduce the ability of TF to induce signalling cascades in endothelial cells via the PAR receptors.

AF2 and AF3 components were more effective than AF1 components—this is mirrored by results for p-selectin inhibition. The highest concentration used was the least effective, possibly reflecting higher stress during the incubation period (significant cell death occurred). IL8 generation was more significantly affected than IL-6 generation, although this may be due to differences in ease of induction of the two cytokines with the concentrations of TF used.

Conclusions:

The results of this experiment show that aqueous tomato extracts reduce the interactions of TF with endothelial cells. We suggest (see Example 4) that this occurs at least in part through its effects on p-selectin expression. These influences on TF-mediated events in endothelial cells imply that thrombin generation as a result of TF/VIIa interaction with endothelial cells or activated platelets will be reduced, preventing activation of the coagulation cascade and clot formation. This is supported by results given in Example 3, where we showed that plasma clotting via the extrinsic system can be suppressed by tomato extract components. We suggest that tomato extracts may have beneficial effects on the key mechanisms influencing venous thrombosis, and in addition, the wider inflammatory system. Effects on IL-6 suggest that liver CRP synthesis could potentially be lowered in vivo. CRP is an independent risk factor for atherosclerosis and CVD. Effects on IL-8 suggest that neutrophil activation may also be suppressed by tomato extract components.

### EXAMPLE 6

### Formulations

### (i) Capsule Formulation

A capsule formulation is prepared by freeze drying a tomato extract as described in Example 1 and filling the resulting freeze dried powder into a hard gelatin capsule shell to give a capsule content of 800 mg per capsule.

### (ii) Capsules Containing Diluted Tomato Extract

To the aqueous tomato extract of Example 1 is added a diluent selected from sucrose, lactose and sorbitol. The resulting mixture is th0-en freeze dried to give a powder which is filled into hard gelatin capsule shells to give a capsule content of 800 mg per capsule (200 mg tomato extract and 600 mg diluent).

(iii) Fruit Drink

The aqueous extract of Example 1 can be added to an orange juice matrix e.g. freshly squeezed orange juice, to give drinks of volumes 50 mL and 200 mL, each of which contains 18 g tomato extract syrup, which is equivalent to the quantity of tomato extract found in 6 fresh tomatoes (total ˜500 g fresh weight).

### EQUIVALENTS

The foregoing examples are presented for the purpose of illustrating the invention and should not be construed as imposing any limitation on the scope of the invention. It will readily be apparent that numerous modification and alterations may be made to the specific embodiments of the invention described above and illustrated in the examples without departing from the principles underlying the invention. All such modifications are intended to be embraced by this application.

1-20. (canceled)

21. A method of preventing, reducing the likelihood of, or inhibiting the initiation of venous thrombosis in a mammalian subject who is subjected to prolonged immobilization, comprising:
providing an effective amount of a tomato extract or an active fraction thereof; and
administering to the subject an effective amount of the tomato extract or active fraction thereof to prevent, reduce the likelihood, or inhibit the initiation of venous thrombosis.

22. The method of claim 21, wherein the tomato extract is an aqueous extract.

23. The method of claim 22, wherein the tomato extract is substantially free of lycopene.

24. The method of claim 22, wherein the aqueous tomato extract is substantially free from water-insoluble particulate material.

25. The method of claim 22, wherein the aqueous tomato extract is substantially free from particulate material.

26. The method of claim 22, wherein the aqueous tomato extract is capable of passing through a 0.2μ filter without loss of solids.

27. The method of claim 21, wherein the tomato extract has been dehydrated to give a water soluble dried extract.

28. The method of claim 21, wherein the tomato extract has been prepared from whole tomato or from a cold-break tomato paste.

29. The method of claim 21, wherein the tomato extract is substantially free of native sugars.

30. A tomato extract or an active fraction thereof for use in preventing, reducing the likelihood of, or inhibiting the initiation of venous thrombosis, or for use in preventing, reducing the likelihood of, or inhibiting the initiation of fibrin clot formation in a vein.

31. The extract of claim 30, wherein the tomato extract is an aqueous extract.

32. The extract of claim 30, wherein the tomato extract is substantially free of lycopene.

33. A method of preventing, reducing the likelihood of, or inhibiting the initiation of fibrin clot formation in a vein in a mammalian subject in need thereof, comprising:
providing an effective amount of the extract or active fraction thereof of claim 30; and
administering the extract to the mammalian subject to prevent, reduce the likelihood of, or inhibit the initiation of fibrin clot formation in the vein.

34. A method of preventing, reducing the likelihood of, or inhibiting the initiation of venous thrombosis in a mammalian subject, comprising:
providing an effective amount of a tomato extract or active fragment thereof of claim 30; and
administering to the subject the effective amount of the tomato extract or active fraction thereof to prevent, reduce the likelihood, or inhibit the initiation of venous thrombosis.

35. A composition comprising a tomato extract or an active fraction thereof use in preventing, reducing the likelihood of, or inhibiting the initiation of venous thrombosis, or for use in preventing, reducing the likelihood of, or inhibiting the initiation of fibrin clot formation in a vein.

36. The composition of claim 35, wherein the tomato extract is an aqueous extract.

37. The composition of claim 35, wherein the tomato extract is substantially free of lycopene.
